(19) United States
(12) Patent Application Publication
(10) Pub. No.: US 2025/0000922 A1
(43) Pub. Date: Jan. 2, 2025

(54) Continue discovery of the root cause of multiple autoimmune disease

(71) Applicant: Majid Kazemi, Tarzana, CA (US)

(72) Inventor: Majid Kazemi, Tarzana, CA (US)

(21) Appl. No.: 18/831,027

(22) Filed: May 6, 2024

Related U.S. Application Data
(63) Continuation-in-part of application No. 14/488,576, filed on Sep. 17, 2014, now abandoned.

(51) Int. Cl. A61K 35/747; A61K 35/00

(52) U.S. Cl. CPC A61K 35/747; A61K 2035/115

(57) ABSTRACT

This invention is done through a real life experiment of mice feeding that close resembles to human genetics with processed, deformed, denatured probiotics of dairy products that human consume daily as well as mice. Mice have similar appetite to dairy product like human do to some level, as mice are addicted to dairy products. The size comparison of a mouse to human is great matter of this experiment since human is 3000 times more than the size of a mouse. What does size mean? It means, If a mouse eat a sample of food with size of a pea for few times and gets disease and die, human must have 100 or thousands times of the same food sample size of the mouse in order to get the diseases. The mice are fed of whole denatured of processed probiotics of dairy products that contain whole a lot of types probiotics without separating any probiotics from dairy products, the same way people consumes dairy product on daily bases without selecting the type of probiotics of dairy. Not all mice got one type of diseases but variety of autoimmune diseases since there were variety of probiotics in processed dairy products, so I got variety of autoimmune diseases since weak organs are more susceptible to diseases in mice and human with signs that similar to human autoimmune diseases like diabetics, Obesity, Psoriasis, Multiple Sclerosis, Muscular dystrophy, Madelung, Paralysis, Parkinson, sudden cardiac arrest, Cataract, and mental disease such as Stress, depression.

### CROSS-REFERENCE TO RELATED APPLICATION

Ser. No. 14/488,576 Continue In Part (CIP)

### BACKGROUND OF INVENTION

Autoimmune diseases are existed for thousands of years on earth, and in the past century only the famous diseases were diagnosed and known to human such as rheumatoid arthritis, diabetics, lupus because of increase of population infected with the diseases. Until today Millions of people are dead from excruciating pain of autoimmune diseases, millions suffering from it, then autoimmune progressed become many types of cancer. No permanent cure has been found for it yet, and all of the therapies are created for it, just temporary relief of symptoms with a lot side effect that gave a ground for establishment of other diseases, and a lot of patients return to square one of disease puzzle since they did not know the root cause of diseases. My discovery of the root causes of many autoimmune diseases will put an end to the multiple autoimmune diseases and reduces the number of cancers in the whole world wide by spreading my knowledge, information of experimental laboratory on mice which it is genetically close to human. Billions of money that spent for researches, drugs, therapies and other development were useless since unknowingly more people around the world are being infected by autoimmune diseases. Also many viruses such as Corona, Covid-19 had huge impact to patients with background of autoimmune diseases deficiency in respiratory, which this virus killed more of this type of people around the world in millions.

### SUMMARY OF THE INVENTION

This invention is done through a real life experiment of mice feeding that close resembles to human genetics with processed, deformed, denatured probiotics of dairy products that human consume daily as well as mice. Mice have similar appetite to dairy product like human do to some level, as mice are addicted to dairy products. The size comparison of a mouse to human is great matter of this experiment since human is 3000 times more than the size of a mouse. What does size mean? It means, If a mouse eat a sample of food with size of a pea for few times and gets disease and die, human must have 100 or thousands times of the same food sample size of the mouse in order to get the diseases. The mice are fed of whole denatured of processed probiotics of dairy products that contain whole a lot of types probiotics without separating any probiotics from dairy products, the same way people consumes dairy product on daily bases without selecting the type of probiotics of dairy. Not all mice got one type of diseases but variety of autoimmune diseases since there were variety of probiotics in processed dairy products, so I got variety of autoimmune diseases since weak organs are more susceptible to diseases in mice and human with signs that similar to human autoimmune diseases like diabetics, Obesity, Psoriasis, Multiple Sclerosis, Muscular dystrophy, Madelung, Paralysis, Parkinson, sudden cardiac arrest, Cataract, and mental disease such as Stress, depression.

### BRIEF DESCRIPTION OF DRAWINGS

Proteins have 4 types of structures in human insulin; primary, secondary, tertiary, and quaternary. FIG. (1)

These structures of proteins change during process of denaturation which the change happen on nucleotides amino acid Adenine (A), Cytosine (C), Guanine (G), Thymine (T). FIG. (2)

Any mutation by any mutagenic of probiotics could shape the structure of the gut micro flora DNA in mice and human cells. FIG. (3)

Protein Synthesis: Translation and Transcription FIG. (4-5)

The DNA damage that happens in many stages of replication FIG. (6-7)

The mechanisms of repairs in our cells FIGS. (8A) and (8B).

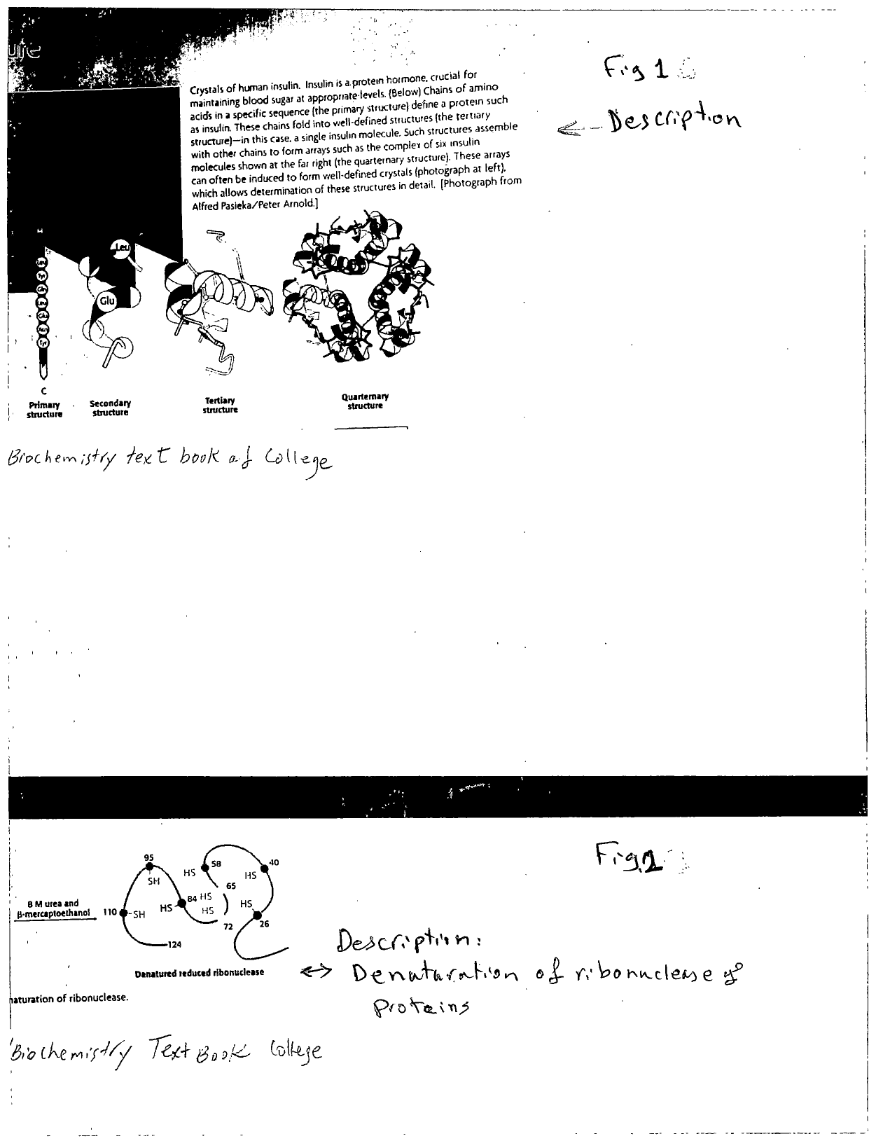

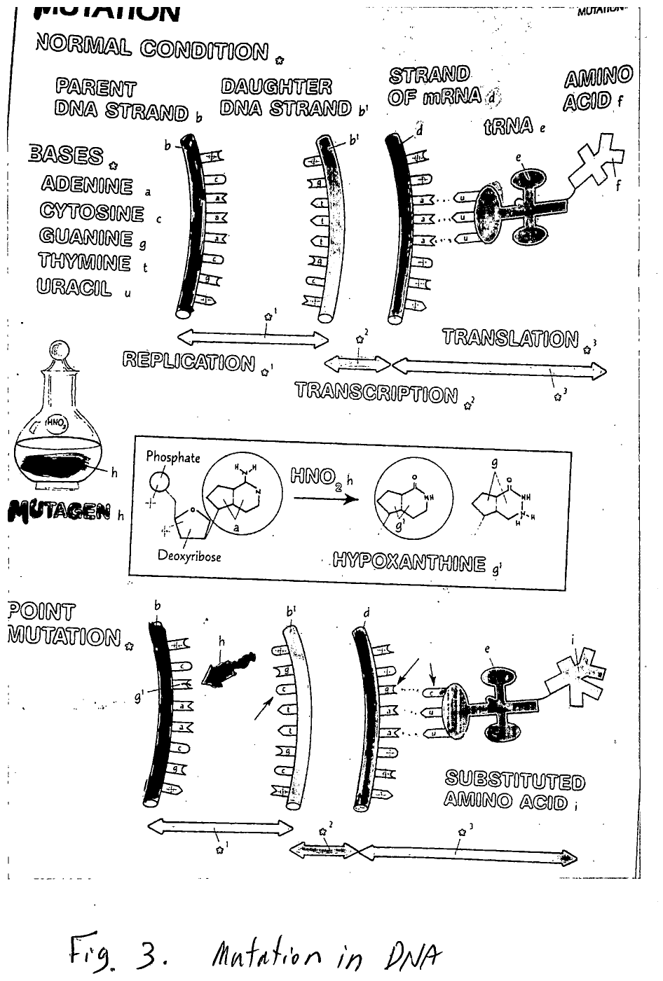

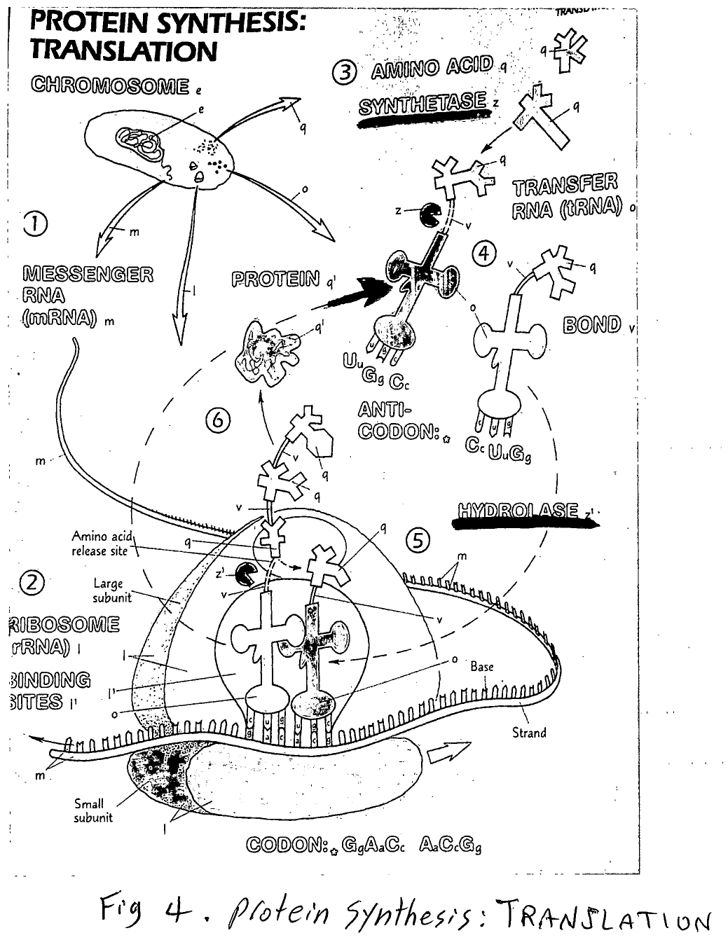

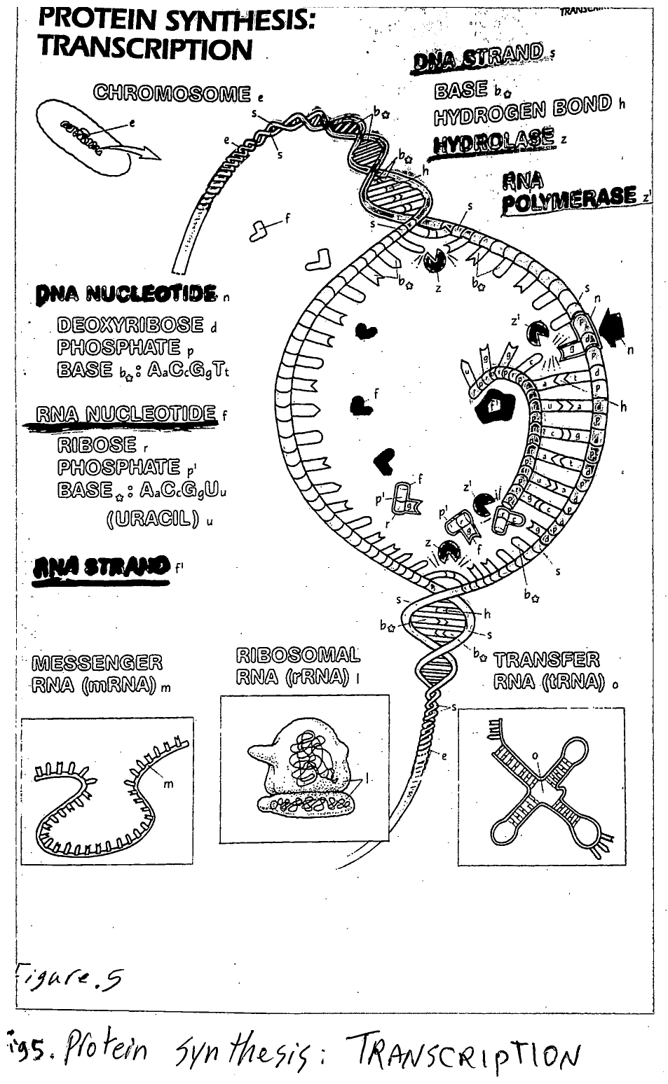

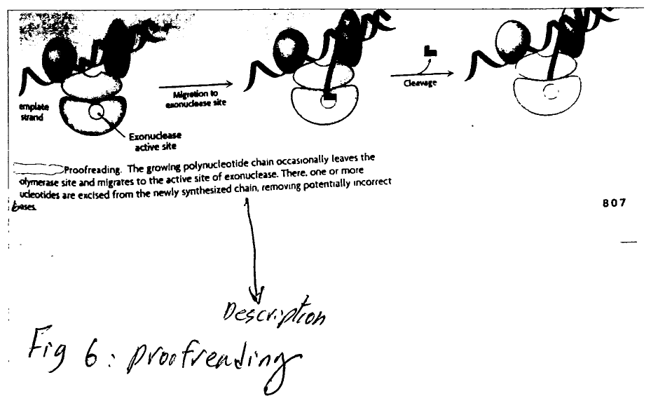

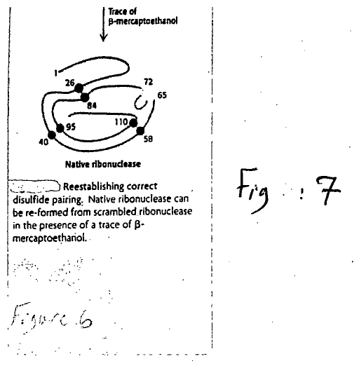

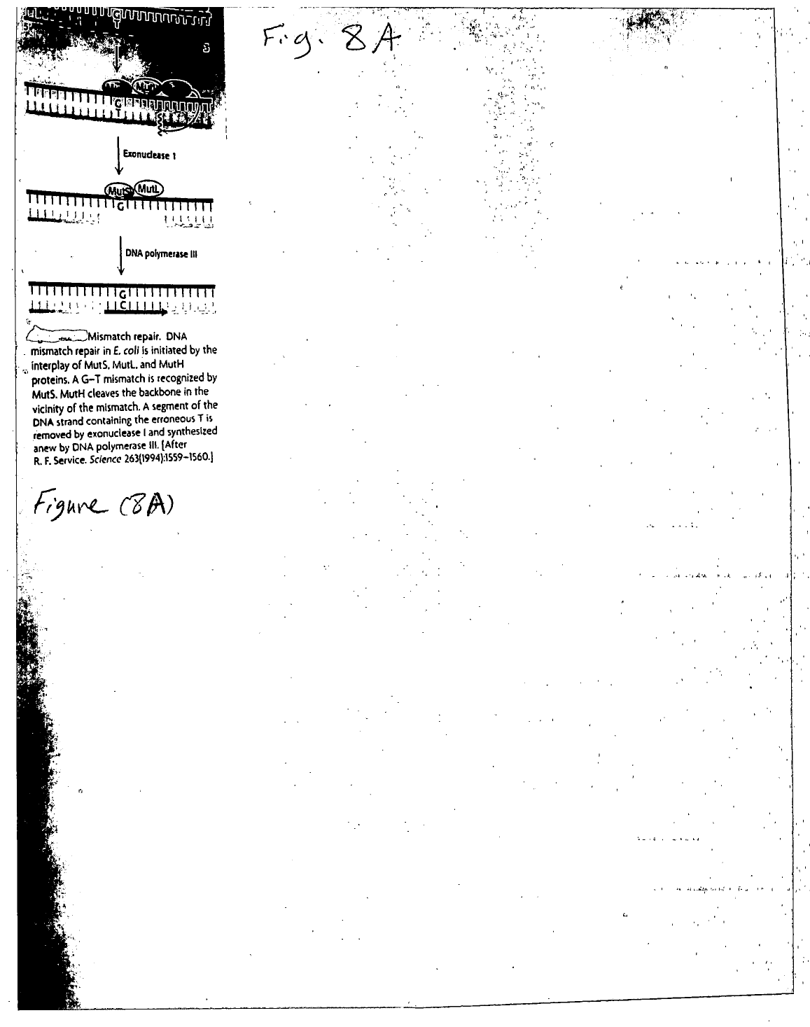

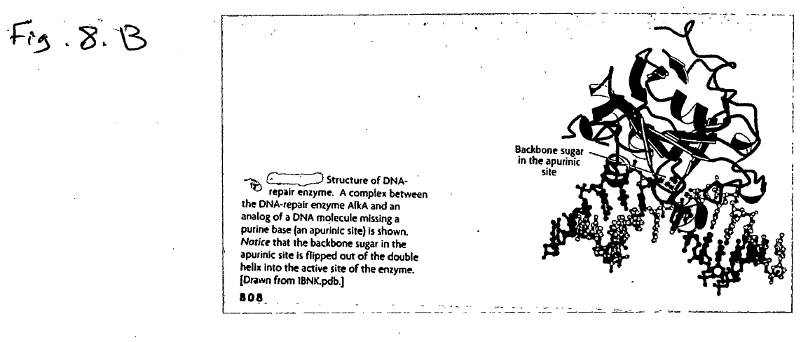

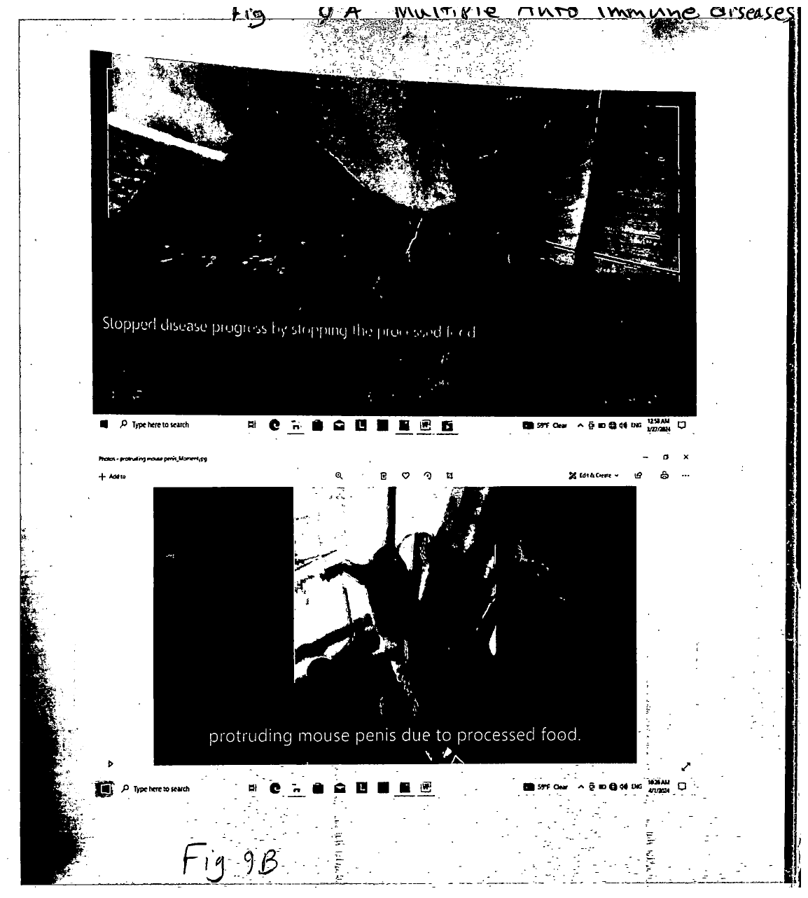

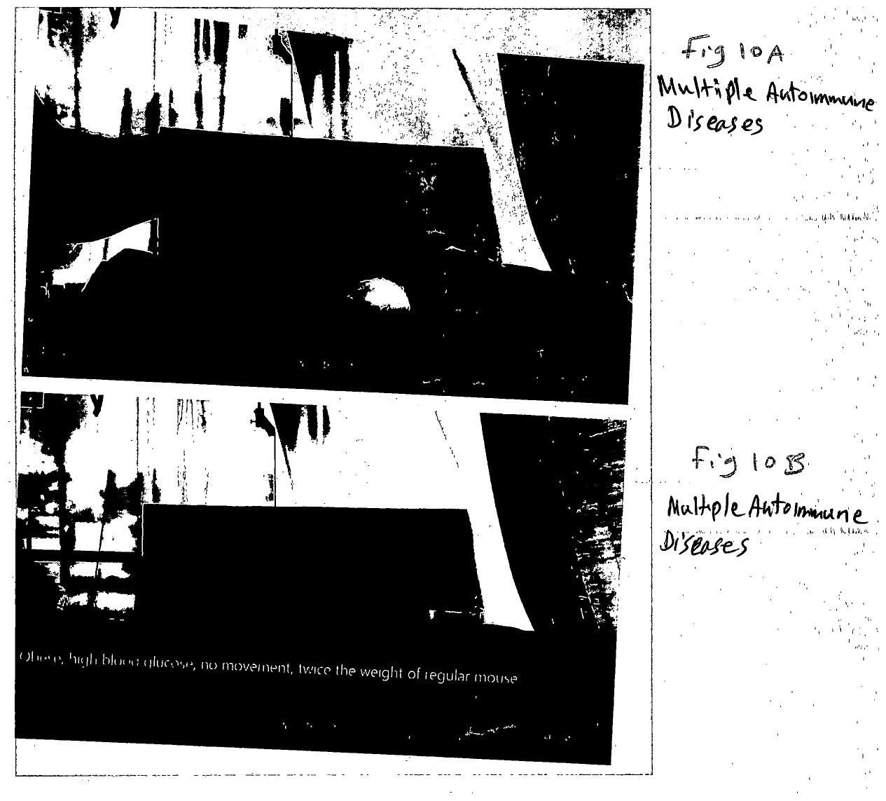

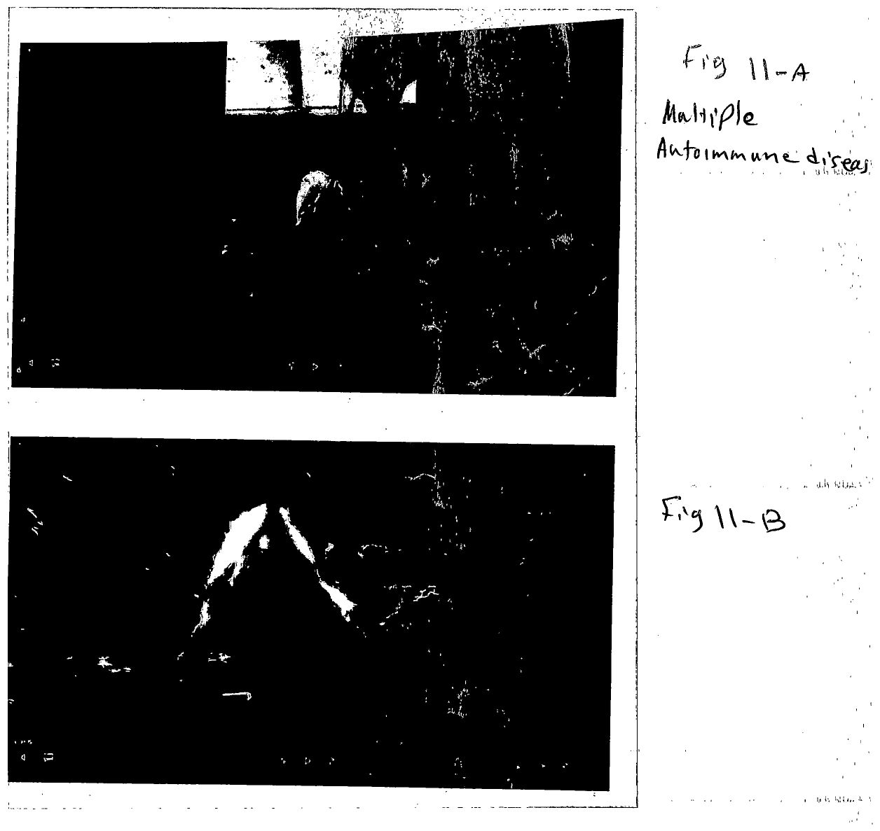

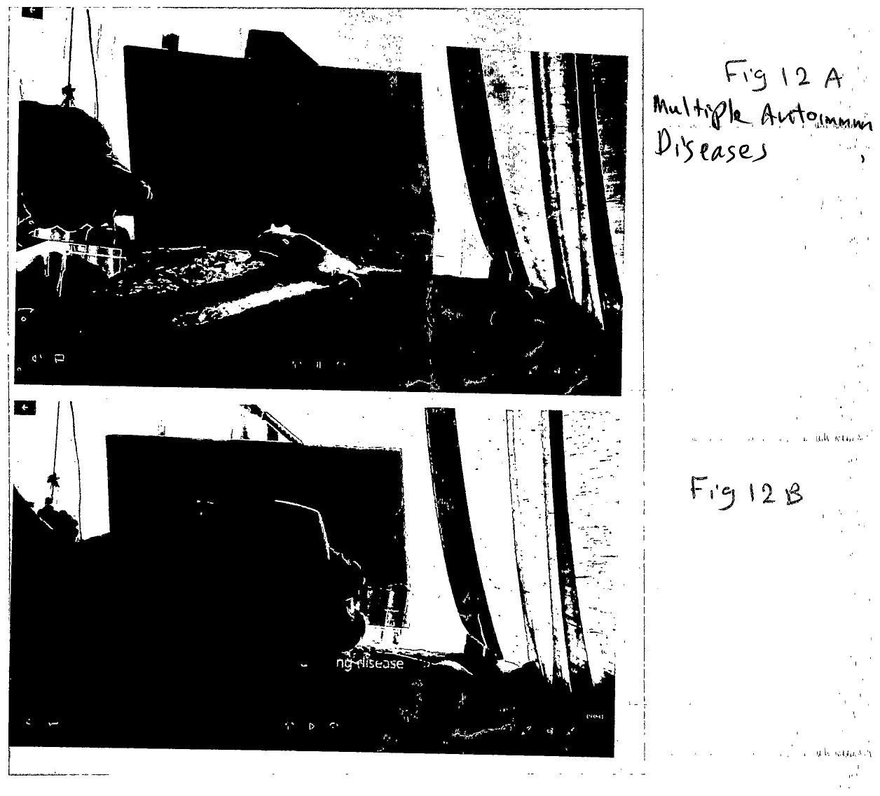

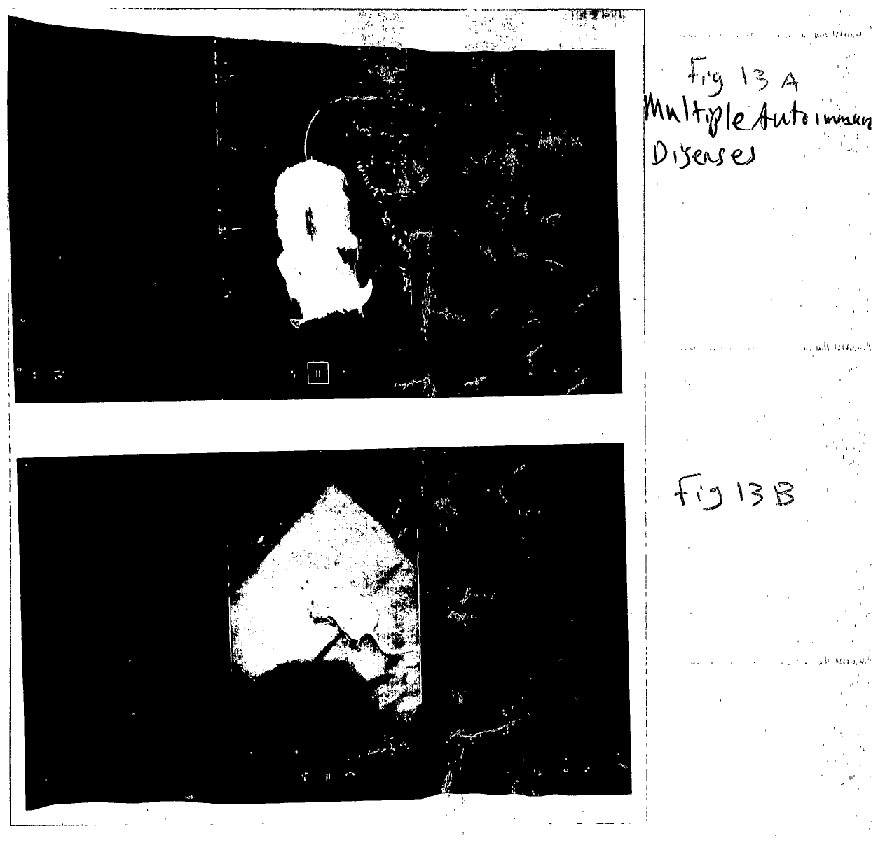

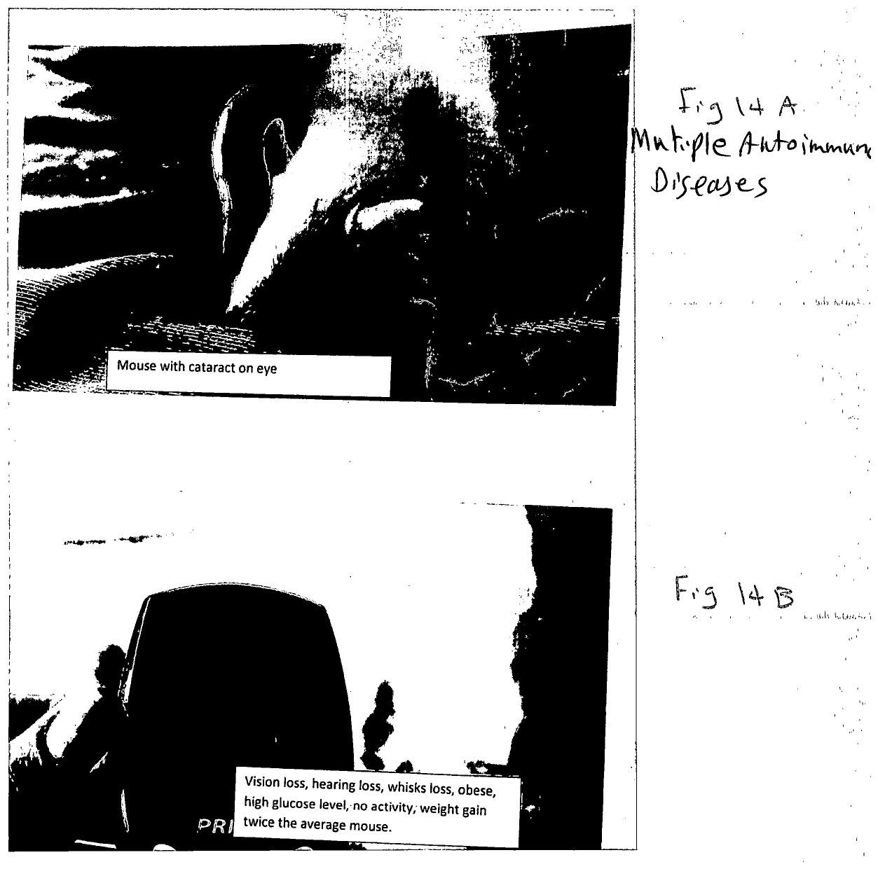

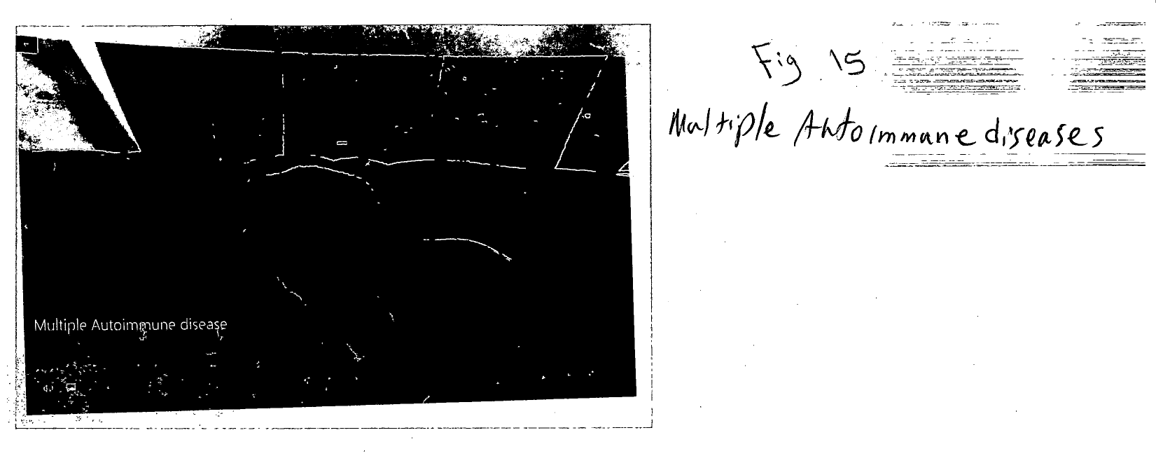

Multiple autoimmune diseases in laboratory mice experiments FIGS. 9 to 15

### DETAILED DESCRIPTION OF THE INVENTION

Mice and human genetically have similar organs, but they have differences; human is 3000 times bigger than mice in weight, size, length, and longer in life line of 65 years than mice which mice shorter in life of 2 years, a size comparison of a mouse with the size of palm of human hand. What do size differences mean for human? It means, in order for a human to get sick with autoimmune diseases or cancer, human must consume hundreds of times or more than the size, and amount of same food material that mice consumes.

The method creation of the root cause of multiple autoimmune diseases that I call them fake autoimmune systems that fight against real autoimmune systems in cells, tissue, and joints. There are collections of all mutated genes of hundreds probiotics family of dairy products; Lactobacillus (L) of acidophilus, L. casei, L. delbrueckii ssp. (bulgaricus) L. cellobiose, L. curvatus, L. lactis, L. fermentum, L. plantarum, L. reuteri, L. brevis, of denatured dairy products milk, cheese, that gradually mutated gene over short period of 3 months in mice, and a year for human with increased consumption of mutated dairy products and for long time with average human life of 75 years with moderate mutated dairy consumption in people which it depends on the % 50 or more of the amount DNA gene mutation in body by any mutagenic agents of acidic, microwave, heating's devices with short time 2-3 minutes temperature below 100 degrees Celsius.

Daily experiment procedure; I feed mice with processed deformed dairy products, denatured DNA probiotics into mice digestive systems through food system especially dairy products with all probiotics that it contain, as human consume these variety probiotics of dairy products daily, without separation of any probiotic from dairy products.

For this experiment; I chose three groups of healthy mice, males, females and control or placebo group which of mixes male and female. These healthy mice were checked up, tested on many conditions; blood glucose level which was 55 to 65, muscular ability by climbing on wire, skin color normal with white fur, eyes color were shiny red, body were slim, hearing were sharp by just snap of fingers would react to direction of sound, vision were perfect and mice follow the object with their eyes, smell of food objects were sharp just by dropping a small food particle or hiding it they would find it quickly, and activities were fast run where they were running all the times except their bed time. Then, purchased all dairy products and all processed dairy food, no poultry, no lame, no beef meat, or fish from local top grocery store products such as; cupcake, garlic cream cheese on top of bread, butter cream filled cake, powder cheese, cheese macaroni and etc.

Male and female mice are given the same processed food on daily bases with warming up the food in microwave for 3 minutes, melting cheese with macaroni, adding other ingredients; salt, sugar, vinegar, while the control group of mice was not given any of the processed food but natural food, vegetarian with no dairy products and no GMO (Genetically Modified Organism).

As scientific surveys shows in a human family of 6 people with autoimmune diseases; if all family members consume the same type of food, not all of family members get the same type of autoimmune diseases but different type disease because of two reasons, one is weak organ and two is random touch of mutated probiotics to micro flora. After three months feeding the mice groups, the signs symptom of problems appears on the mice male and female body with lost whisks, rashes of Psoriasis disease on back of skin, losing fur, then blood glucose level (bgl) increase measured from 50 to 100 then going over 200 (bgl). One mouse got nerve issues or tremor that made it difficult for normal walking that looked like shaking. One mouse got paralysis on lower body that was not able to walk on feet, like crawling with hands. One mouse got Parkinson disease where mouse was not able to hold food particle in hand while eating it. One mouse got cataract in eyes with vision problem that could not see the location of water container or food location then became blind. One muse got hearing problem that could not hear or show reaction to the direction of sound. One mouse became obese with over weight of 45. grams which it was above normal 2 to 3 times the average laboratory mice 32. grams. One mouse got cardiac arrest with sudden death. 2 mice got Madelung disease with large deposit of fat on neck, arms, under belly and feet. One mouse got MS disease which it was not moving correctly, difficulty climbing wire and walking. Multiple autoimmune diseases FIGS. 9 to 15

This experiment and my previous one of root cause of autoimmune disease with application/patent proves that many of my theories are correct;
1) The scientific survey that not all family members get the same type of autoimmune disease even though if they all had the same type of food consumption since the mutated probiotic act differently with different organ.
2) The theory that one or few of denatured, mutated dairy probiotics could hit randomly one or multiple organs that create multiple types of autoimmune diseases.
3) The root cause of diabetics is not sugar, and the root cause of cardiovascular disease is not salt.
4) The GMO (genetically modified organism) product that engineered with bacteria is harmful to any living existence human or nonhuman.
5) Scientists believe that autoimmune system attacks its own cells and organs which it is not correct of real autoimmune systems, the one that do that is the fake autoimmune systems disease that is created of unrepaired mismatch damaged amino acids during protein synthesis of amino acids in cells in number of millions.
6) Inflammation rises due to conflict of real autoimmune systems with fake autoimmune systems.

As the denatured, mutated probiotics of dairy products enter to digestion system in the gut, the deformed DNA probiotics inject their deformed DNA sequences in to the DNA sequences of mice or human gut micro flora and it makes transformation. The world medical scientists proved that, these probiotics of dairy have, proteins, genes, DNA, and nucleotides amino acid. Proteins have 4 types of structures in human insulin; primary, secondary, tertiary, and quaternary. FIG. (1)

These structures of proteins change during process of denaturation which the change happen on nucleotides amino acid Adenine (A), Cytosine (C), Guanine (G), Thymine (T). FIG. (2)

As these mutated, changed nucleotides go through mechanism of DNA synthesis, the systems would not recognize a matching pair for A,C,G,T so the system dumps the changed, deformed amino acids as junk amino acid which later these are become the Fake Autoimmune systems diseases in organs then after accumulation of these junk amino acids to threshold level, then the fighting start between real autoimmune system and fake autoimmune systems disease (FAISD), then human or mice feel the inflammation in some region or organ.

Any mutation by any mutagenic of probiotics could shape the structure of the gut micro flora DNA in mice and human cells. FIG. (3)

If all hundreds of human gut micro flora of lactobacillus, Candida, Streptococcus, Helicobacter Pylori, Peptostreptococcus, Large intestinal; Clostridium coccids, Leptum, Fusobacterium, Bacteroides sp, Coliforms, Bifid bacterium, Enter bacteria, Enterococcus, Faecalis, Peptococcus, Peptostreptococcus, Ruminococcus of humans in numbers of billions are mutated multiple times by mutagenic in to human digestive system then it becomes defective gene, causing fake autoimmune systems diseases.

The following mutagenic agents change the genomic structure of micro flora gut genes and organs in mice and human when the probiotics become deformed by heating, radiation material such as microwave oven, x-Ray, dry freeze, exposure to low temperature, and by acid chemical strong acidic food with low PH, and mutagenic, carcinogenic products, heavy metals mercury, lead, arsenic. These gene mutations create alteration in amino acids sequences which produce abnormal function and autoimmune diseases.

When we look at the detail molecular level, human and mice consume protein, our body cell process the amino acids through protein synthesis which it requires the translation of nucleotides sequences into amino acid sequences. The basic of protein synthesis is the same pathway throughout all cells animal or plant but the process is slightly different in plants which plants use sunlight in process of photosynthesis. This method of protein synthesis is known to scientists that a protein is synthesized in the direction of amino-to-carboxyl end of growing peptide chain. After break down of protein to amino acids, the amino acids arrive at growing chain in activated form as aminoacyl-t RNAs, which it attach to the carboxyl group of amino acid to 3′ end of t-RNA, then the linkage of amino acid to its corresponding tRNA is catalyzed by an enzyme that is called aminacyl-t RNA Synthetase. The process of protein synthesis with translation must be accurate with lowest error, otherwise junk DNA, and junk amino acid accumulation would be the result of the protein synthesis which it is the start of accumulation of fake autoimmune systems diseases. Protein Synthesis: Translation and Transcription FIG. (4-5).

Does protein synthesis mechanism make mistake? Yes, as scientist proved it, absolutely correct, just by looking how many types of autoimmune diseases exist now. What is the rate of accuracy for protein synthesis? According to scientific research, for small protein with 100 amino acids should be less than 0.366, for 300 amino acids is 0.049 and for 1000 amino acids should be 0.000. The error frequency must not exceed the approximate of 10-4 to produce lager protein effectively. The formula for probability (P) of forming a protein with no error depend on (n) the number of amino acids and (e) the frequency of insertion of a wrong amino acid: P=(1−(e))ⁿ

Many researches are done by scientists about the DNA damage that happens in many stages of replication, which it can be as simple as disincorporation of a single base or complex forms in a base chemical modification, in cross linking between two strands of DNA double helix or in the breaks in one or both of the phosphodiester backbones. The results of DNA damages could be cell death, cell miss-transformation, change in DNA sequences that could be inherited by future generation and blockage of DNA replication process. It is found out by researchers that as we get DNA damages we also have repair mechanisms that correct the problem by proofreading DNA systems but it is not always complete, since the repair mechanism misses some process of synthesis that we see fake autoimmune systems disease appearance in the future.

The damage in DNA replication process is the simplest one, that a wrong base enter the double helix of Watson-Crick base pair and get incorporated in to the base which then forming a non-Watson-Crick base pair. This mismatch is not corrected by the repair mechanism which then causes damage to DNA double helix which it is a mutagenic that can cause permanent changes in DNA sequences. There are other problems could happen in DNA strands including wrong insertion, deletion, and wrong breaks in different location of DNA double helix. Also the replicative enzyme polymerase can stop functioning, or leaving untouched a damaged template at all without replication completion. FIG. (6-7)

A study research of mutagenic shows nucleotides bases can be damaged in many ways by mutagenic agents such as; oxidizing, alkylating, x-ray, microwave, light and temperature. The mutagenic agent of oxidizing like hydroxyl radical can react with guanine (G) to form 8-oxoguanine then it cannot pair with cytosine (C) in DNA replication but it pairs with adenine (A) which it is unmatched. Another mutagenic is the deamination process that also damages DNA, which adenine (A) would be deaminated to form hypoxanthine, this is a mutagenic that pairs with cytosine (C) instead of thymine (T). Similar unmatched pairs bases occur when other mutagenic agents such as alkylating, X-ray, microwave, UV light, temperature react with DNA double helix.

While we have many mechanisms of repairs in our cells, protein synthesis and genes but continuous over entry of billions of mismatch nucleotides makes the mechanism miss mange the synthesis process, as result of that we get fake autoimmune systems disease and then a pathway to cancer in future.

It is proven by researchers that in autoimmune system mechanism, we have the replicative DNA polymerase that correct mismatches DNA. Mismatch repair system that use two proteins Mut S, Mut L and an enzyme that is called endonuclease Mut H to repair the mismatch nucleotides. FIG. (8A)

Direct repair system is the photochemical cleavage of pyrimidine dimers that is done by the enzyme DNA Photolyase that comes from photo reactivating enzyme.

Based excision repair is another repair systems that is done by E. coli enzyme AlkA on a modified base of 3-methyladenine, the binding of this enzyme AlkA to damage DNA flips the affected base out of DNA double helix and into the active site of the enzyme then cleaving the glycosidic bond by enzyme glycosylase for releasing damaged nucleotide base. FIG. (8B)

Many types of diseases and cancers come from defective mechanism of repair systems of DNA, as we know cancers are caused by mutation of genes associated with growth control. As defects increase in DNA repair systems so the number of autoimmune diseases increase as well as cancers, for example of Xeroderma-pigmentation a rare skin disease, or hereditary non-polyposis colorectal cancer and many more.

How does fake autoimmune system function in body? It acts like confused autoimmune systems, when it gets to a threshold level, it degrade, damages the amino acid of cells, it carries microbes to less protected site organs that no defensive mechanism exist which it causes malfunction of that organ. When there is an injury site in muscle, it reach to the site and causes severe inflammation pain.

### INTRODUCTION OF CLAIMS TO EXAMINER

This research, experiment is done as whole in real life example of mice that genetically similar to human in matter of digestive organs, consumption of foods especially the whole dairy products that contain multiple probiotics. We consume processed dairy products without separating the probiotics of the food, so the mice are fed with processed probiotics of dairy products without separating any of probiotics from the containment. Dairy products have multiple probiotics, when we process and denature the dairy products that have multiple probiotics we get denaturation of protein structure and mutate the gene of nucleotides of amino acids in mice or human. Then repeating complex of denatured, mutated multiple probiotics entry to mice or human guts which then it hits any internal organs that cause it to malfunction. Any mutated of probiotic species could hit in random any organ continuously of mice or human which after a period of time that is depends on the amount of denatured nucleotides, it causes fake autoimmune system diseases. So it is unspecified species of many mutated probiotics of dairies could damage the organs of mice or human. It is like the game of Russian Rolette where ever the dice hit the number on rotating table. It is not a specified probiotic that hit an organ. As scientific surveys show in a family of 6 people with multiple autoimmune diseases that they consume the same food but not all of family members get the same type of autoimmune diseases but different type. My research proves that multiple autoimmune diseases arise from multiple mutated probiotics. The following claims for this research are based on above experiment.

Notes: These three claims are dependent since in the process of experiment.

1. The method creation of the root cause of multiple autoimmune diseases that I call them fake autoimmune systems disease that fight against real autoimmune systems cells, tissue, and joints are the collections of all mutated genes of hundreds probiotics family of dairy products; Lactobacillus of acidophilus, L. casei, L. delbrueckii ssp. (bulgaricus) L. cellobiose, L. curvatus, L. lactis, L. fermentum, L. plantarum, L. reuteri, L. brevis, of denatured dairy products milk, cheese, that gradually mutated over short period of 3 months in mice and a year with increased consumption of mutated dairy products in human, then in long time with average human life of 75 years with moderate mutated dairy consumption in people which it depends on the % 50 or more of the amount DNA gene mutation in body by any mutagenic agents of acidic, microwave, heating's devices with temperature below 100 degrees Celsius.

2. In continuation of process claim 1, the method creation of the root cause of multiple autoimmune diseases, mutated genes of whole humans or mice gut micro flora by; lactobacillus, Candida, Streptococcus, Helicobacter Pylori, Peptostreptococcus, Large intestinal; Clostridium Coccoides, Leptum, Fusobacterium, Bacteroides sp, Coliforms, Bifid bacterium, Enterobacteria, Enterococcus, Faecalis, Bifidobacteria, Peptococcus, Peptostreptococcus, Ruminococcus and hundred more, may change, mutate the gene of micro flora, then it would become fake autoimmune system disease, with defective gene in human or mice micro flora which it creates autoimmune disease then it is pathway to cancers.

3. In continuation of process claims 1 and 2, the method creation of the root cause of multiple autoimmune diseases, are few denatured, mutated dairy probiotics that could hit randomly one or multiple organs that create multiple types of autoimmune diseases.
